(19) United States
(12) Patent Application Publication
(10) Pub. No.: US 2018/0280294 A1
(43) Pub. Date: Oct. 4, 2018

(54) THERAPEUTIC PUTTY HAVING ANALGESICS AND/OR COUNTERIRRITANTS

(71) Applicant: DEPCO, INC., Hauppauge, NY (US)

(72) Inventors: M. Gregory Minuto, Smithtown, NY (US); Stephen Mlcoch, Sound Beach, NY (US)

(21) Appl. No.: 15/937,179

(22) Filed: Mar. 27, 2018

Related U.S. Application Data
(60) Provisional application No. 62/620,686, filed on Jan. 23, 2018.
(60) Provisional application No. 62/484,056, filed on Apr. 11, 2017.
(60) Provisional application No. 62/479,607, filed on Mar. 31, 2017.

(51) Int. Cl. A61K 9/06; A61K 47/34; A61K 31/167; A61K 31/165; A61K 31/60; A61K 31/045; A61K 47/10; A61K 31/618; A61K 31/125; A61K 36/28; A61K 31/10; A61K 31/4152; A61K 45/06; A61K 31/137

(52) U.S. Cl. CPC A61K 9/06; A61K 47/34; A61K 31/167; A61K 31/165; A61K 31/60; A61K 31/045; A61K 31/137; A61K 31/618; A61K 31/125; A61K 36/28; A61K 31/10; A61K 31/4152; A61K 45/06; A61K 47/10

(57) ABSTRACT

A therapeutic putty for use in treating patients who have hand maladies and which helps alleviate the patient's pain when undergoing hand therapeutic exercises includes a silicone putty base and at least one of an analgesic and a counterirritant as an ingredient thereof and mixed with the silicone putty base.

### CROSS-REFERENCE TO RELATED APPLICATIONS

This application is related to U.S. Provisional Application Ser. Nos. 62/479,607, 62/484,056 and 62/620,686, filed on Mar. 31, 2017, Apr. 11, 2017 and Jan. 23, 2018, respectively, each entitled “Therapeutic Putty Having Analgesics And/Or Counterirritants”, the disclosure of each of which is incorporated herein by reference and on which priority is hereby claimed.

### BACKGROUND OF THE INVENTION

### Field of the Invention

The present invention relates to therapeutic and exercise putties.

### Description of the Prior Art

Many orthopedic and physical therapists treat patients who have hand maladies. These maladies cover a wide range of issues from people who have strength issues to those who have issues with arthritis. Much of the rehabilitation is done through exercise. One of the ways the exercise is performed is with therapeutic putty.

Therapeutic putty is much like Dow Corning's Q2-3233 which is a boronated silicone elastomer. These elastomers may vary in viscosity and color.

One issue which has been noted is the problem of pain management during exercise or subsequent to exercise. The invention disclosed herein provides a means to help resolve this problem and to relieve pain associated with hand therapeutic exercises.

### OBJECTS AND SUMMARY OF THE INVENTION

It is an object of the present invention to provide a therapeutic putty which is manipulated by a patient and which includes one or more ingredients which act to help alleviate the patient's pain when undergoing hand therapeutic exercises.

The invention is directed to a therapeutic or exercise putty in which either an analgesic or counterirritant or anesthetic ingredient is mixed with the base putty.

These and other objects, features and advantages of the present invention will be apparent from the following detailed description of illustrative embodiments thereof.

### DETAILED DESCRIPTION OF THE PREFERRED EMBODIMENTS

In accordance with the present invention, a boronated silicone elastomer, such as Part No. Q2-3233, manufactured by The Dow Chemical Company of Midland, Mich., includes ingredients mixed therewith which act as an analgesic or counterirritant or anesthetic.

The following preferred formulations for such a therapeutic putty, containing ingredients which act as an analgesic or counterirritant or anesthetic, are provided below, stated in approximate parts by weight of the therapeutic putty:

| | Embodiment 1 | Embodiment 2 | Embodiment 3 |
|---|---|---|---|
| 1-Dow Corning Q2-3233 | 99 pts. to 80 pts. | 85 pts. to 10 pts. | 70 pts. |
| Lidocaine (Generically, 2- | 1 pt. to 20 pts. | 15 pts. to 90 pts. | 30 pts. |
| (Dimethylamino)-N-(2,6- | | | |
| Dimethylphenyl) acetamide) | | | |
| 2-Dow Corning Q2-3233 | 99 pts. to 80 pts. | 85 pts. to 10 pts. | 70 pts. |
| Capsaicin (Generically, 6-Nonenamide, | 1 pt. to 20 pts. | 15 pts. to 90 pts. | 30 pts. |
| N-((4-Hydroxy-3- | | | |
| methoxyphenyl)methyl)-8-methyl-,(E)) | | | |
| 3-Dow Corning Q2-3233 | 99 pts. to 80 pts. | 85 pts. to 10 pts. | 70 pts. |
| Salicylate (aspirin) (Generically, 2- | 1 pt. to 20 pts. | 15 pts. to 90 pts. | 30 pts. |
| Hydroxybenzoic acid, monosodium salt; | | | |
| Chemical Name: Salicylic Acid) | | | |
| 4-Dow Corning Q2-3233 | 99 pts. to 80 pts. | 85 pts. to 10 pts. | 70 pts. |
| Menthol (Generically, Hexahydrothymol; | 1 pt. to 20 pts. | 15 pts. to 90 pts. | 30 pts. |
| (1alpha,2beta,5alpha)-5-Methyl-2-(1- | | | |
| methylethyl)cyclohexanol) | | | |
| 5-Dow Corning Q2-3233 | 99 pts. to 80 pts. | 85 pts. to 10 pts. | 70 pts. |
| Methylsalicylate (Generically, Oil of | 1 pt. to 20 pts. | 15 pts. to 90 pts. | 30 pts. |
| Wintergreen (Synthetic); 2- | | | |
| Hydroxybenzoic acid methyl ester) | | | |
| 6-Dow Corning Q2-3233 | 99 pts. to 80 pts. | 85 pts. to 10 pts. | 70 pts. |
| Camphor (Chemical Name: (DL)- | 1 pt. to 20 pts. | 15 pts. to 90 pts. | 30 pts. |
| Camphor)) | | | |
| 7-Dow Corning Q2-3233 | 99 pts. to 80 pts. | 85 pts. to 10 pts. | 70 pts. |
| Arnica Montana (Biological Definition: | 1 pt. to 20 pts. | 15 pts. to 90 pts. | 30 pts. |
| The oil extracted from the dried flower | | | |
| heads of Arnica Montana by infusion in | | | |
| sunflower oil (Helianthus Annuus Seed | | | |
| Oil) at a ratio of 1:10.) | | | |
| 8-Dow Corning Q2-3233 | 99 pts. to 80 pts. | 85 pts. to 10 pts. | 70 pts. |
| Trolamine Salicylate | 1 pt. to 20 pts. | 15 pts. to 90 pts. | 30 pts. |
| 9-Dow Corning Q2-3233 | 99 pts. to 80 pts. | 85 pts. to 10 pts. | 70 pts. |
| Methyl Sulfonyl Methane (Product | 1 pt. to 20 pts. | 15 pts. to 90 pts. | 30 pts. |
| Name: Methylsulfonylmethane) | | | |

One or more non-narcotic, or narcotic, analgesic types of ingredients that are effective for pain relief may be used in substitution for the analgesics or counterirritants set forth in the list provided previously for use in the therapeutic putty of the present invention. The analgesics which are preferably used should be chosen that are best for pain or inflammatory relief and in accordance with the putty use technique.

Furthermore, methanol-based treatment products that are often sold as topical irritant or pain relief products, and which act to relieve pain relatively quickly, may be used in the therapeutic putty of the present invention.

More specifically, non-narcotic analgesics which may possibly be used as an ingredient in the therapeutic putty of the present invention include, but are not limited to, and are identified by their generic names as Acetaminophen and Metamizole (which is believed to be banned in certain countries, including the United States). Non-steroidal anti-inflammatory drugs (NSAIDs) may also be used as ingredients of the therapeutic putty of the present invention, and include, but are not limited to, and are identified by their generic names as Aspirin, Diclofenac, Dexibuprofen, Diflunisal, Etodolac, Fenoprofen, Flufenamic acid, Flurbiprofen, Ibuprofen, Indomethacin, Ketoprofen, Ketorolac, Lomoxicam, Loxoprofen, Meclofenamic acid, Mefenamic acid, Meloxicam, Nabumetone, Naproxen, Oxaprozin, Piroxicam, Salsalate, Sulindac, Tenoxicam, Tolmetin and Tolfenamic acid.

Narcotic analgesics, also commonly referred to as opiates, opioid analgesics, or narcotics, may also be used as an ingredient in the therapeutic putty of the present invention. Such narcotic analgesics include COX-2 inhibitors, including, but not limited to, and identified by their generic names as Celecoxib, Rofecoxib, Valdecoxib, Parecoxib and Etoricoxib (certain of these ingredients may have been withdrawn from the market entirely or withdrawn from use in the United States).

Such narcotic analgesics further include opioid analgesics, including, but not limited to, and identified by their generic names as Buprenorphine, Butorphanol, Codeine, Hydrocodone, Hydromorphone, Levorphanol, Meperidine, Methadone, Morphine, Nalbuphine, Oxycodone, Oxymorphone, Pentazocine and Propoxyphene. Diclofenac is a most effective nonsteroidal anti-inflammatory drug.

Such narcotic analgesics additionally include, but are not limited to, and are identified by their generic names as Tapentadol and Tramadol.

Also, combination analgesics, which contain one or more types of analgesics, may also possibly be used as an ingredient in the therapeutic putty of the present invention. Such analgesic combinations include, but are not limited to, and are identified by their generic names as Acetaminophen and Aspirin; Acetaminophen and Aspirin and Caffeine; Acetaminophen and Aspirin and Caffeine and Salicylamide; Acetaminophen and Caffeine and Magnesium and Salicylate; Acetaminophen and Caffeine and Phenyltoloxamine and Salicylamide; Acetaminophen and Caffeine and Isometheptene; Acetaminophen and Butalbital; Acetaminophen and Salicylamide; Acetaminophen and Caffeine; Acetaminophen and Phenyltoloxamine; Aspirin and Caffeine; Aspirin and Caffeine and Salicylamide; Aspirin and Caffeine and Orphenadrine; Aspirin and Meprobamate; Butalbital and Acetaminophen and Caffeine; Butalbital and Aspirin and Caffeine; Butalbital and Acetaminophen and Caffeine and Codeine; Butalbital and Aspirin and Caffeine and Codeine; Buprenorphine and Naloxone; Codeine and Acetaminophen; Codeine and Aspirin; Codeine and Ibuprofen; Codeine and Acetaminophen and Doxylamine; Dihydrocodeine and Acetaminophen and Caffeine; Dihydrocodeine and Aspirin and Caffeine; Hydrocodone and Acetaminophen; Hydrocodone and Aspirin; Hydrocodone and Ibuprofen and Oxycodone and Acetaminophen; Oxycodone and Aspirin; Oxycodone and Ibuprofen; Propoxyphene and Acetaminophen; Propoxyphene and Aspirin; Propoxyphene and Aspirin and Caffeine; Pentazocine and Acetaminophen; Pentazocine and Aspirin; and Tramadol and Acetaminophen.

Some ingredients such as menthol may need to be dissolved in a solvent like alcohol to be more available for the patient.

The therapeutic putty of the present invention, such as disclosed in the above formulations, will aid in reducing obstructions to patient compliance to exercise due to the reduction in swelling and pain.

Although illustrative embodiments of the present invention have been described herein, it is to be understood that the invention is not limited to those precise embodiments, and that various other changes and modifications may be effected therein by one skilled in the art without departing from the scope or spirit of the invention.

What is claimed is:

1. A therapeutic putty, which comprises:
a silicone putty base; and
at least one of an analgesic and a counterirritant as an ingredient thereof and mixed with the silicone putty base.

2. A therapeutic putty as defined by claim 1, wherein the silicone putty base is Part No. Q2-3233 manufactured by The Dow Chemical Company of Midland, Mich.

3. A therapeutic putty as defined by claim 1, wherein the at least one of an analgesic and a counterirritant ingredient includes lidocaine (2-(Dimethylamino)-N-(2,6-Dimethylphenyl) acetamide).

4. A therapeutic putty as defined by claim 3, wherein the lidocaine is in the range of about 1 part to about 20 parts by weight of the therapeutic putty, and the silicone putty base is in the range of about 99 parts to about 80 parts by weight of the therapeutic putty.

5. A therapeutic putty as defined by claim 3, wherein the lidocaine is in the range of about 15 parts to about 90 parts by weight of the therapeutic putty, and the silicone putty base is in the range of about 85 parts to about 10 parts by weight of the therapeutic putty.

6. A therapeutic putty as defined by claim 5, wherein the lidocaine is about 30 parts by weight of the therapeutic putty, and the silicone putty base is about 70 parts by weight of the therapeutic putty.

7. A therapeutic putty as defined by claim 1, wherein the at least one of an analgesic and a counterirritant ingredient includes capsaicin (6-Nonenamide, N-((4-Hydroxy-3-methoxyphenyl)methyl)-8-methyl-,(E)).

8. A therapeutic putty as defined by claim 7, wherein the capsaicin is in the range of about 1 part to about 20 parts by weight of the therapeutic putty, and the silicone putty base is in the range of about 99 parts to about 80 parts by weight of the therapeutic putty.

9. A therapeutic putty as defined by claim 7, wherein the capsaicin is in the range of about 15 parts to about 90 parts by weight of the therapeutic putty, and the silicone putty base is in the range of about 85 parts to about 10 parts by weight of the therapeutic putty.

10. A therapeutic putty as defined by claim 9, wherein the capsaicin is about 30 parts by weight of the therapeutic putty, and the silicone putty base is about 70 parts by weight of the therapeutic putty.

11. A therapeutic putty as defined by claim 1, wherein the at least one of an analgesic and a counterirritant ingredient includes salicylate (2-Hydroxybenzoic acid, monosodium salt).

12. A therapeutic putty as defined by claim 11, wherein the salicylate is in the range of about 1 part to about 20 parts by weight of the therapeutic putty, and the silicone putty base is in the range of about 99 parts to about 80 parts by weight of the therapeutic putty.

13. A therapeutic putty as defined by claim 11, wherein the salicylate is in the range of about 15 parts to about 90 parts by weight of the therapeutic putty, and the silicone putty base is in the range of about 85 parts to about 10 parts by weight of the therapeutic putty.

14. A therapeutic putty as defined by claim 13, wherein the salicylate is about 30 parts by weight of the therapeutic putty, and the silicone putty base is about 70 parts by weight of the therapeutic putty.

15. A therapeutic putty as defined by claim 1, wherein the at least one of an analgesic and a counterirritant ingredient includes menthol (Hexahydrothymol; (1alpha,2beta,5alpha)-5-Methyl-2-(1-methylethyl)cyclohexanol).

16. A therapeutic putty as defined by claim 15, wherein the menthol is in the range of about 1 part to about 20 parts by weight of the therapeutic putty, and the silicone putty base is in the range of about 99 parts to about 80 parts by weight of the therapeutic putty.

17. A therapeutic putty as defined by claim 15, wherein the menthol is in the range of about 15 parts to about 90 parts by weight of the therapeutic putty, and the silicone putty base is in the range of about 85 parts to about 10 parts by weight of the therapeutic putty.

18. A therapeutic putty as defined by claim 17, wherein the menthol is about 30 parts by weight of the therapeutic putty, and the silicone putty base is about 70 parts by weight of the therapeutic putty.

19. A therapeutic putty as defined by claim 15, which further comprises:
a solvent, the menthol being dissolved in the solvent.

20. A therapeutic putty as defined by claim 19, wherein the solvent includes alcohol.

21. A therapeutic putty as defined by claim 1, wherein the at least one of an analgesic and a counterirritant ingredient includes methylsalicylate (Oil of Wintergreen (Synthetic); 2-Hydroxybenzoic acid methyl ester).

22. A therapeutic putty as defined by claim 21, wherein the methylsalicylate is in the range of about 1 part to about 20 parts by weight of the therapeutic putty, and the silicone putty base is in the range of about 99 parts to about 80 parts by weight of the therapeutic putty.

23. A therapeutic putty as defined by claim 21, wherein the methylsalicylate is in the range of about 15 parts to about 90 parts by weight of the therapeutic putty, and the silicone putty base is in the range of about 85 parts to about 10 parts by weight of the therapeutic putty.

24. A therapeutic putty as defined by claim 23, wherein the methylsalicylate is about 30 parts by weight of the therapeutic putty, and the silicone putty base is about 70 parts by weight of the therapeutic putty.

25. A therapeutic putty as defined by claim 1, wherein the at least one of an analgesic and a counterirritant ingredient includes camphor ((DL)-Camphor).

26. A therapeutic putty as defined by claim 25, wherein the camphor is in the range of about 1 part to about 20 parts by weight of the therapeutic putty, and the silicone putty base is in the range of about 99 parts to about 80 parts by weight of the therapeutic putty.

27. A therapeutic putty as defined by claim 25, wherein the camphor is in the range of about 15 parts to about 90 parts by weight of the therapeutic putty, and the silicone putty base is in the range of about 85 parts to about 10 parts by weight of the therapeutic putty.

28. A therapeutic putty as defined by claim 27, wherein the camphor is about 30 parts by weight of the therapeutic putty, and the silicone putty base is about 70 parts by weight of the therapeutic putty.

29. A therapeutic putty as defined by claim 1, wherein the at least one of an analgesic and a counterirritant ingredient includes Arnica Montana.

30. A therapeutic putty as defined by claim 29, wherein the Arnica Montana is in the range of about 1 part to about 20 parts by weight of the therapeutic putty, and the silicone putty base is in the range of about 99 parts to about 80 parts by weight of the therapeutic putty.

31. A therapeutic putty as defined by claim 29, wherein the Arnica Montana is in the range of about 15 parts to about 90 parts by weight of the therapeutic putty, and the silicone putty base is in the range of about 85 parts to about 10 parts by weight of the therapeutic putty.

32. A therapeutic putty as defined by claim 31, wherein the Arnica Montana is about 30 parts by weight of the therapeutic putty, and the silicone putty base is about 70 parts by weight of the therapeutic putty.

33. A therapeutic putty as defined by claim 1, wherein the at least one of an analgesic and a counterirritant ingredient includes trolamine salicylate.

34. A therapeutic putty as defined by claim 33, wherein the trolamine salicylate is in the range of about 1 part to about 20 parts by weight of the therapeutic putty, and the silicone putty base is in the range of about 99 parts to about 80 parts by weight of the therapeutic putty.

35. A therapeutic putty as defined by claim 33, wherein the trolamine salicylate is in the range of about 15 parts to about 90 parts by weight of the therapeutic putty, and the silicone putty base is in the range of about 85 parts to about 10 parts by weight of the therapeutic putty.

36. A therapeutic putty as defined by claim 35, wherein the trolamine salicylate is about 30 parts by weight of the therapeutic putty, and the silicone putty base is about 70 parts by weight of the therapeutic putty.

37. A therapeutic putty as defined by claim 1, wherein the at least one of an analgesic and a counterirritant ingredient includes Methyl Sulfonyl Methane.

38. A therapeutic putty as defined by claim 37, wherein the Methyl Sulfonyl Methane is in the range of about 1 part to about 20 parts by weight of the therapeutic putty, and the silicone putty base is in the range of about 99 parts to about 80 parts by weight of the therapeutic putty.

39. A therapeutic putty as defined by claim 37, wherein the Methyl Sulfonyl Methane is in the range of about 15 parts to about 90 parts by weight of the therapeutic putty, and the silicone putty base is in the range of about 85 parts to about 10 parts by weight of the therapeutic putty.

40. A therapeutic putty as defined by claim 39, wherein the Methyl Sulfonyl Methane is about 30 parts by weight of the therapeutic putty, and the silicone putty base is about 70 parts by weight of the therapeutic putty.

41. A therapeutic putty as defined by claim 1, wherein the at least one of an analgesic and a counterirritant ingredient includes a non-narcotic analgesic selected from the group consisting of Acetaminophen and Metamizole.

42. A therapeutic putty as defined by claim 1, wherein the at least one of an analgesic and a counterirritant ingredient includes a non-steroidal anti-inflammatory drug selected from the group consisting of Aspirin, Diclofenac, Dexibuprofen, Diflunisal, Etodolac, Fenoprofen, Flufenamic acid, Flurbiprofen, Ibuprofen, Indomethacin, Ketoprofen, Ketorolac, Lomoxicam, Loxoprofen, Meclofenamic acid, Mefenamic acid, Meloxicam, Nabumetone, Naproxen, Oxaprozin, Piroxicam, Salsalate, Sulindac, Tenoxicam, Tolmetin and Tolfenamic acid.

43. A therapeutic putty as defined by claim 1, wherein the at least one of an analgesic and a counterirritant ingredient includes a narcotic analgesic having a COX-2 inhibitor and selected from the group consisting of Celecoxib, Rofecoxib, Valdecoxib, Parecoxib and Etoricoxib.

44. A therapeutic putty as defined by claim 1, wherein the at least one of an analgesic and a counterirritant ingredient includes a narcotic, opioid analgesic selected from the group consisting of Buprenorphine, Butorphanol, Codeine, Hydrocodone, Hydromorphone, Levorphanol, Meperidine, Methadone, Morphine, Nalbuphine, Oxycodone, Oxymorphone, Pentazocine and Propoxyphene.

45. A therapeutic putty as defined by claim 1, wherein the at least one of an analgesic and a counterirritant ingredient includes a narcotic analgesic selected from the group consisting of Tapentadol and Tramadol.

46. A therapeutic putty as defined by claim 1, wherein the at least one of an analgesic and a counterirritant ingredient includes a combination of analgesics or counterirritants, the combination being selected from the group consisting of Acetaminophen and Aspirin; Acetaminophen and Aspirin and Caffeine; Acetaminophen and Aspirin and Caffeine and Salicylamide; Acetaminophen and Caffeine and Magnesium and Salicylate; Acetaminophen and Caffeine and Phenyltoloxamine and Salicylamide; Acetaminophen and Caffeine and Isometheptene; Acetaminophen and Butalbital; Acetaminophen and Salicylamide; Acetaminophen and Caffeine; Acetaminophen and Phenyltoloxamine; Aspirin and Caffeine; Aspirin and Caffeine and Salicylamide; Aspirin and Caffeine and Orphenadrine; Aspirin and Meprobamate; Butalbital and Acetaminophen and Caffeine; Butalbital and Aspirin and Caffeine; Butalbital and Acetaminophen and Caffeine and Codeine; Butalbital and Aspirin and Caffeine and Codeine; Buprenorphine and Naloxone; Codeine and Acetaminophen; Codeine and Aspirin; Codeine and Ibuprofen; Codeine and Acetaminophen and Doxylamine; Dihydrocodeine and Acetaminophen and Caffeine; Dihydrocodeine and Aspirin and Caffeine; Hydrocodone and Acetaminophen; Hydrocodone and Aspirin; Hydrocodone and Ibuprofen and Oxycodone and Acetaminophen; Oxycodone and Aspirin; Oxycodone and Ibuprofen; Propoxyphene and Acetaminophen; Propoxyphene and Aspirin; Propoxyphene and Aspirin and Caffeine; Pentazocine and Acetaminophen; Pentazocine and Aspirin; and Tramadol and Acetaminophen.
